(19) United States
(12) Patent Application Publication
(10) Pub. No.: US 2026/0243750 A1
(43) Pub. Date: Aug. 20, 2026

(54) METHOD AND APPARATUS FOR MONITORING WATER QUALITY

(71) Applicant: Lee RASHKIN, Dalton, NH (US)

(72) Inventor: Lee RASHKIN, Dalton, NH (US)

(21) Appl. No.: 19/441,503

(22) Filed: Jan. 6, 2026

Related U.S. Application Data
(60) Provisional application No. 63/742,011, filed on Jan. 6, 2025.

(51) Int. Cl. G01N 33/18; E03F 5/10

(52) U.S. Cl. CPC G01N 33/18; E03F 5/105; E03F 2201/20

(57) ABSTRACT

The disclosed method enables versatile water quality monitoring across applications like septic systems, wastewater facilities, stormwater management, agricultural runoff, and recreational water settings. By leveraging advanced sensors and imaging for continuous data collection and analysis, the method facilitates informed decision-making, autonomous operation, and preventive maintenance. It ensures regulatory compliance, reduces failure risks, and optimizes resource use. Stakeholders, including property owners, service providers, engineers, and researchers, benefit from streamlined operations, enhanced designs, and real-time data access. The method contributes to public health by preventing contamination, aligns with environmental goals through early pollutant detection, and generates economic value through cost savings and operational efficiency. Adaptable for fixed, temporary, or portable setups, the method supports decentralized data aggregation, offering significant advancements over traditional water monitoring systems.

### BACKGROUND

### Technical Field

The present disclosure relates to the field of water quality monitoring, and in particular, relates to a method and apparatus for monitoring water quality in water treatment systems.

### CROSS-REFERENCE TO RELATED APPLICATIONS

The present disclosure claims benefit of U.S. Patent Publication No. 63/742,011, titled METHOD AND APPARATUS FOR MONITORING WATER QUALITY, filed Jan. 6, 2025. The entire contents are hereby incorporated by reference herein.

### Description of the Related Art

Maintaining water quality is essential for public health, environmental sustainability, and industrial operations. Traditional water quality monitoring technologies primarily rely on manual sampling and laboratory analysis, which are labor-intensive, time-consuming, and prone to errors due to sample degradation during transport. These conventional approaches often involve the collection of discrete samples at predetermined intervals, with water quality parameters such as pH, turbidity, dissolved oxygen, conductivity, and temperature being measured off-site. While effective in controlled environments, these methods fail to provide real-time or continuous data, limiting their ability to detect rapid changes in water quality. This results in significant data gaps, hindering proactive maintenance and increasing the risk of system failures, high repair costs, and environmental hazards.

Historically, water quality monitoring systems have relied on simple probes and standalone sensors to measure individual parameters directly within the water source. These systems often employ single-function sensors, such as pH electrodes, turbidity meters, or dissolved oxygen probes, which relay data to local control units for analysis. While effective for basic measurements, such systems are limited by their inability to provide comprehensive insights into water quality dynamics. Additionally, they are prone to issues such as biofouling and sensor drift, requiring frequent maintenance, recalibration, and manual cleaning to ensure accurate and reliable measurements. These limitations reduce the effectiveness of traditional monitoring methods, particularly in environments where continuous and precise data collection is critical.

In industrial applications, such as wastewater treatment and cooling water management, legacy monitoring systems often employ basic flow-through cells with fixed sensors. These setups are often rigid, difficult to upgrade, and limited in scalability, making them unsuitable for dynamic environments where flexibility is required. Furthermore, they rely solely on electrochemical measurements that cannot provide insight into the presence of suspended solids or particulates.

In recent years, advancements in water quality monitoring have led to the development of networked sensing platforms that aim to provide more comprehensive data collection. However, many of these platforms are unsuitable for remote or off-grid locations. These systems typically feature static configurations, which reduce flexibility and adaptability to varying monitoring environments and response to rapid changes in water quality. For instance, monitoring agricultural runoff, stormwater, or recreational water facilities requires flexible solutions that can be deployed rapidly and efficiently. Furthermore, such platforms often lack the robustness and reliability needed for continuous real-time data transmission, particularly in large-scale or distributed monitoring networks, thereby diminishing their effectiveness in managing water quality over extensive geographic areas.

Despite the advancements, existing water quality monitoring technologies remain inadequate in environments requiring continuous, real-time analysis, rapid detection of contaminants, and autonomous operation. Therefore, there remains a need for an improved method and apparatus for water quality monitoring that overcomes the above-mentioned drawbacks of traditional technologies.

### BRIEF SUMMARY

One or more embodiments relate to methods and apparatuses for monitoring water quality in a variety of applications, such as septic systems, municipal wastewater facilities, industrial wastewater treatment plants, stormwater management systems, agricultural runoff monitoring, and recreational water environments such as pools and hot tubs. In an embodiment, the apparatus includes advanced sensors and imaging modules to enable continuous data collection and analysis. Based on the analysis, the apparatus facilitates informed decision-making, autonomous operations, and optimized performance across diverse water quality management scenarios.

The modular design of the apparatus presents significant benefits for stakeholders in various sectors. For regulatory authorities, the apparatus facilitates automated compliance with water quality standards, reducing the burden of manual inspections and lowering the risk of penalties. Property owners benefit from reduced failure risks and maintenance costs, while service providers can streamline their operations with real-time monitoring and early issue detection. Preventive maintenance is enhanced across residential, commercial, educational, medical, and recreational facilities, ensuring consistent functionality and minimizing disruptions. For engineers and designers, integrating high-quality data enables improvements in design and development processes. Researchers and academics gain access to a robust platform for advancing water quality technologies and exploring new methodologies for water management.

In an embodiment, the disclosed apparatus also contributes to broader societal and environmental objectives. By preventing water contamination, it improves public health outcomes and mitigates risks associated with unsafe water in residential and recreational settings. Early detection of contaminants aligns with environmental conservation goals, reducing the impact of pollutants on ecosystems. Additionally, the economic value generated through cost savings, operational efficiencies, and resource optimization makes it a valuable solution for municipalities, businesses, and individuals. Adaptable configurations, including fixed, temporary, and portable installations, further enhance its applicability. Decentralized networking capabilities enable autonomous data aggregation and improve system-wide visibility, representing a marked advancement over conventional water monitoring methods.

In an embodiment, the apparatus offers significant advantages for various stakeholders, including ensuring automated regulatory compliance, mitigating failure risks for property owners, and simplifying maintenance for service providers. Further, the apparatus supports preventive maintenance in diverse environments such as residential homes, businesses, hospitals, schools, and recreational facilities, guaranteeing consistent and reliable operation while minimizing the risk of malfunctions. Furthermore, the apparatus facilitates flexible integration for engineers and designers, offering enhanced capabilities for design optimization and product development through access to high-quality, real-time data. For academics and researchers, the apparatus offers an ideal platform for further advancements in water management technologies. Further, the apparatus facilitates improved community health by preventing water contamination, aligns with environmental conservation objectives through early detection of contaminants, and generates substantial economic value through cost savings, operational efficiencies, and more effective resource management. Furthermore, the apparatus may be configured for fixed, temporary, or portable installations, ensuring adaptability to a wide range of monitoring needs. Moreover, the apparatus supports decentralized networking, enabling autonomous aggregation of data, which represents a significant advancement over traditional monitoring technologies.

An embodiment of the present disclosure relates to the method for water quality monitoring across a wide range of applications, including septic systems, municipal wastewater facilities, industrial treatment plants, stormwater management systems, agricultural runoff monitoring, and recreational water environments such as pools and hot tubs. The method employs advanced sensors and imaging techniques to enable continuous data collection and analysis. By processing the data, the method facilitates informed decision-making, autonomous operations, and enhanced performance in managing water quality.

The modular nature of the method offers significant advantages to stakeholders. For regulatory authorities, the method ensures automated compliance with water quality standards, reducing reliance on manual inspections and lowering the risk of non-compliance penalties. Property owners benefit from reduced maintenance costs and minimized failure risks, while service providers streamline their operations through real-time monitoring and early issue detection. The method enhances preventive maintenance in various settings, including residential, commercial, medical, educational, and recreational facilities, ensuring uninterrupted functionality and minimizing disruptions. Furthermore, engineers and designers can leverage high-quality data generated by the method to optimize design processes and advance product development. Researchers and academics gain access to a platform for exploring innovative water management technologies.

The method also addresses broader societal and environmental goals. By detecting contaminants early, the method helps prevent water contamination, improving public health outcomes and reducing risks in residential and recreational water settings. Its ability to align with environmental conservation objectives mitigates the impact of pollutants on ecosystems. Additionally, the method generates substantial economic value by reducing operational costs, improving efficiency, and optimizing resource utilization. The method supports fixed, temporary, or portable configurations, making it adaptable to a wide range of monitoring needs. Its decentralized networking capabilities allow for autonomous data aggregation, significantly enhancing system-wide visibility and representing a marked improvement over traditional water monitoring methods.

The features and advantages of the subject matter here will become more apparent in light of the following detailed description of selected embodiments, as illustrated in the accompanying FIGURES. As will be realized, the subject matter disclosed is capable of modifications in various respects, all without departing from the scope of the subject matter. Accordingly, the drawings and the description are to be regarded as illustrative in nature.

### BRIEF DESCRIPTION OF THE DRAWINGS

The present subject matter will now be described in detail with reference to the drawings, which are provided as illustrative examples of the subject matter so as to enable those skilled in the art to practice the subject matter. Notably, the FIGURES and examples are not meant to limit the scope of the present subject matter to a single embodiment, but other embodiments are possible by way of interchange of some or all of the described or illustrated elements and, further, wherein:

FIG. 1 illustrates an exemplary smart baffle for efficient water quality monitoring, in accordance with an embodiment of the present disclosure;

FIG. 2 illustrates an exemplary operation of the smart baffle for monitoring wastewater in a septic tank, in accordance with an embodiment of the present disclosure;

FIG. 3A and FIG. 3B illustrate exemplary operations of the smart baffle for monitoring wastewater in septic tank having chamber baffle walls, in accordance with an embodiment of the present disclosure;

FIG. 4 illustrates an aerobic treatment system having a baffle system to optimize wastewater quality, in accordance with an embodiment of the present disclosure;

FIG. 5 illustrates an exemplary Smart Inline Networked Canister (SINC), in accordance with an embodiment of the present disclosure;

FIG. 6 illustrates an exemplary operation of a removable top and bottom of the SINC, in accordance with an embodiment of the present disclosure;

FIG. 7 illustrates an exemplary Smart Distribution Box (SMWT D-Box) managing the flow of wastewater, in accordance with an embodiment of the present disclosure;

FIG. 8 illustrates an exemplary solar-powered buoy system for remote water quality monitoring and management, in accordance with an embodiment of the present disclosure; and

FIG. 9 illustrates a flowchart of a method for monitoring water quality, in accordance with an embodiment of the present disclosure.

Other features of embodiments of the present disclosure will be apparent from accompanying drawings and detailed description that follows.

### DETAILED DESCRIPTION

The detailed description set forth below in connection with the appended drawings is intended as a description of exemplary embodiments in which the presently disclosed disclosure can be practiced. The term “exemplary” used throughout this description means “serving as an example, instance, or illustration,” and should not necessarily be construed as preferred or advantageous over other embodiments. The detailed description includes specific details for providing a thorough understanding of the presently disclosed disclosure. However, it will be apparent to those skilled in the art that the presently disclosed disclosure may be practiced without these specific details. In some instances, well-known structures and devices are shown in block diagram form in order to avoid obscuring the concepts of the presently disclosed disclosure.

Embodiments of the present disclosure include various steps, which will be described below. The steps may be performed by hardware components or may be embodied in machine-executable instructions, which may be used to cause a general-purpose or special-purpose processor programmed with the instructions to perform the steps. Alternatively, steps may be performed by a combination of hardware, software, and/or firmware.

Embodiments of the present disclosure may be provided as a computer program product, which may include a non-transitory, machine-readable storage medium tangibly embodying thereon instructions, which may be used to program the computer (or other electronic devices) to perform a process. The machine-readable medium may include, but is not limited to, fixed (hard) drives, semiconductor memories, such as Read Only Memories (ROMs), Programmable Read-Only Memories (PROMs), Random Access Memories (RAMs), Erasable PROMs (EPROMs), Electrically Erasable PROMs (EEPROMs), flash memory or other types of media/machine-readable medium suitable for storing electronic instructions (e.g., computer programming code, such as software or firmware).

Various methods described herein may be practiced by combining one or more non-transitory, machine-readable storage media containing the code according to the present disclosure with appropriate standard computer hardware to execute the code contained therein. An apparatus for practicing various embodiments of the present disclosure may involve one or more computers (or one or more processors within the single computer) and storage systems containing or having network access to a computer program(s) coded in accordance with various methods described herein, and the method steps of the disclosure could be accomplished by modules, routines, subroutines, or subparts of a computer program product.

The terms “connected” or “coupled” and related terms are used in an operational sense and are not necessarily limited to a direct connection or coupling. Thus, for example, two devices may be coupled directly, or via one or more intermediary media or devices. As another example, devices may be coupled in such a way that information can be passed there between, while not sharing any physical connection. Based on the disclosure provided herein, one of ordinary skill in the art will appreciate a variety of ways in which connection or coupling exists in accordance with the aforementioned definition.

Further, the term “module” may be software or hardware particularly programmed to receive an input, perform one or more processes using the input, and provide an output. The input, output, and processes performed by various modules will be apparent to one skilled in the art based on the present disclosure.

If the specification states a component or feature “may,” “can,” “could,” or “might” be included or have a characteristic, that particular component or feature is not required to be included or have the characteristic.

As used in the description herein and throughout the claims that follow, the meaning of “a,” “an,” and “the” includes plural reference unless the context dictates otherwise. Also, as used in the description herein, the meaning of “in” includes “in” and “on” unless the context dictates otherwise.

The phrases “in an embodiment,” “according to one embodiment,” and the like generally mean the particular feature, structure, or characteristic following the phrase is included in at least one embodiment of the present disclosure and may be included in more than one embodiment of the present disclosure. Importantly, such phrases do not necessarily refer to the same embodiment.

It will be appreciated by those of ordinary skill in the art that the diagrams, schematics, illustrations, and the like represent conceptual views or processes illustrating systems and methods embodying this disclosure. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this disclosure. Those of ordinary skill in the art further understand that the exemplary hardware, software, processes, methods, and/or operating systems described herein are for illustrative purposes and, thus, are not intended to be limited to any particular name.

Embodiments of the present disclosure relate to a method and apparatus to provide a water quality monitoring system for efficient and reliable data collection across a wide range of environments. The method incorporates a defined sampling area strategically optimized through the placement of detection components such as sensors, cameras, probes, and lighting systems. The components are positioned relative to the flow path of the water (also termed as liquid) to ensure that the collected samples are representative of the water's overall and/or specific quality. The sampling area may be designed to create ideal conditions for accurate measurements by taking into account factors such as lighting, fluid dynamics, flow rate, turbulence, and particulate distribution. The sampling area can be customized in shape, with options for cylindrical, conical, box-like, plate-like, or spherical configurations, and its size can be adjusted to suit specific applications ranging from portable units to larger industrial setups. Further, the components are constructed from non-corrosive materials such as metals, polymers, rubber, and fiberglass.

The monitoring method is highly flexible and can be deployed in a variety of configurations, including fixed installations within pipes or tanks, free-floating or suspended setups, and robotic or modular options such as pill cams and mechanical robots. Inline structures can also be used to channel the fluid flow, creating stable sampling volumes. Multi-purpose canisters and chambers may be employed as inline, submerged, plumbed, or mobile units equipped with plumbing attachments, lighting, and sensor systems for comprehensive, flexible monitoring solutions.

The imaging system, a key component of the invention, is available in both basic and advanced versions. It supports the detection of turbidity and other visual indicators of water quality, with advanced versions providing enhanced features for comprehensive imaging in challenging conditions. The imaging system utilizes high-end hardware, such as CMOS or CCD sensors, which offer superior noise reduction and perform well in low-light conditions. These sensors capture high-resolution images with advanced features like phase detection autofocus and High Dynamic Range (HDR), allowing for detailed analysis. Multi-lens configurations, such as wide, ultra-wide, and telephoto lenses, enhance the system's imaging flexibility by providing optical zoom and depth perception, ensuring that the system can adapt to various visual requirements. The system also supports adaptive lighting systems, including UV and infrared modules, which improve visibility in low-light or submerged environments, as well as advanced image stabilization and multi-directional camera configurations to ensure precise assessments of water quality.

The imaging system is further enhanced by the integration of chemically enhanced imaging, which utilizes specific chemical agents or indicators that react with substances in the water to improve contrast and visual clarity. This feature is particularly useful in environments where traditional imaging might not provide sufficient differentiation. Additionally, a range of advanced imaging technologies can be incorporated to further improve the system's capabilities. These include Lidar for precise distance measurements and the creation of 3D models, hyperspectral imaging to detect a wide range of substances by analyzing their spectral signatures, and thermal imaging to detect temperature variations associated with biological activity or contamination. Other imaging technologies include fluorescence imaging for detecting biological substances, sonar imaging for low-visibility environments, and structured light scanning to measure depth and shape. The system also supports polarized imaging to reduce glare and 360-degree panoramic cameras that offer a full view without the need for repositioning.

The lighting system in the invention ensures that imaging can take place in diverse and challenging environments. Basic lighting options include on-demand flash lighting, LED arrays, and infrared lighting for low-light and submerged environments, with adjustable intensity to enhance contrast and clarity. The advanced modular lighting system includes adaptive reflective surfaces, UV lighting for biological detection, and polarized lighting to reduce glare. Additional lighting options include fiber optic illumination for narrow spaces, chemically enhanced lighting to highlight specific substances, and color-specific illumination to detect contaminants.

The method also includes robust data transfer and communication capabilities, supporting a range of data transmission options, including Bluetooth, Wi-Fi, LoRaWAN, cellular networks, and wired connections. These features provide flexibility in data collection, processing, and integration with centralized or local storage systems. Data can be transmitted to remote devices, such as mobile phones or computers, facilitating real-time monitoring and analysis. The system supports hybrid and decentralized networks, enabling multiple units to aggregate localized data into a central database, thus improving system-wide visibility and predictive maintenance. In some cases, the system or device may include a processor, microcontroller, or other computer, and in some other cases, the sensors and other devices of the device or system may be in wired or wireless communication with a remote microcontroller or other computer. In those cases, the microcontroller can be configured to process or transmit data gathered by the sensors and other devices to various other devices, including a user device of a system owner or administrator, or to a service provider to schedule maintenance.

The method facilitates decentralized monitoring, with multiple units functioning cohesively to aggregate localized data into a central database, improving system-wide visibility and predictive maintenance. Integrated locating systems using Bluetooth, Wi-Fi, LoRaWAN, or other wireless frequencies allow for easy identification of each unit, simplifying setup and maintenance. The method also facilitates centralized mapping to aggregate location data for efficient tracking, distribution, and maintenance planning.

The power options are versatile, supporting a range of energy sources such as solar panels, battery systems, wind power, gas, or hardwired electrical connections, ensuring adaptability across different installations. The invention also includes several secondary components to enhance its functionality. The gravity-driven flow mechanism maintains consistent sampling without mechanical intervention, ensuring an “always-full” sampling area and reducing maintenance complexity. The use of a gravity-flow sampling chamber ensures optimal sampling consistency by utilizing fluid dynamics to minimize clogging and maintain a stable sampling environment. For example, a modular water quality monitoring canister used in wastewater treatment systems may rely on gravity to direct water into the chamber, ensuring a constant flow for continuous and accurate sampling. In a particular embodiment, the gravity-flow method may facilitate the canister to passively and continuously capture representative water samples from the effluent without requiring mechanical pumps, shunts, valves or additional intervention, thereby simplifying the sampling process and reducing the need for frequent maintenance.

The components are coated with anti-microbial and anti-stick treatments, preventing biofilm buildup and extending operational longevity. The system is designed to be fully submersible, with waterproof and anti-corrosive features that ensure reliable operation in wastewater and other aquatic environments. The canister's modular ports and “smart chamber” design allow for versatile attachment points, enabling the canister to function as a standalone sampling device or be integrated into a larger monitoring system. Various attachment options, such as adhesive bonding, magnetic coupling, physical clamping, screws, straps, or friction fittings, ensure secure installation in a wide range of environments.

The method includes integrating drawing and/or pumping mechanisms, such as pumps or diaphragms, with plumbing attachments for drawing, holding, and discharging samples as needed. The pumping mechanism may ensure precise and reliable sample handling within the system, enabling automatic or manual activation depending on the requirements of the process. Pumps, whether peristaltic, diaphragm, or other types, are used to pump water from specific points, such as household to the system. Further, the pump may be utilized to pump out the treated water.

The plumbing attachments associated with these mechanisms are designed to securely connect to the system's flow paths, ensuring that samples are captured and transported efficiently without leakage or loss. These attachments may include flexible tubing, quick-connect fittings, or dedicated valves that facilitate easy disconnection for maintenance or cleaning. The system can also be equipped with automated valves or flow controllers to manage the timing and volume of sample collection, ensuring consistent sample integrity and preventing cross-contamination. By integrating these pumping and drawing mechanisms, the method optimizes the accuracy, flexibility, and efficiency of sample collection and discharge, making it adaptable to a wide range of applications, including continuous monitoring and batch sampling scenarios.

Additionally, the method includes integrating disinfection and self-cleaning mechanisms using UV light, chlorine, ozone, or other chemical treatments to ensure water quality and reduce manual intervention. Further, the self-cleaning UV mechanisms may be integrated with UV treatment systems to ensure that the water is effectively pre-treated before disinfection. The integration may allow for the removal or reduction of particulate matter, organic contaminants, and microbial load. By improving the clarity and quality of the water through pre-treatment, the UV system may function more efficiently, ensuring that the water is thoroughly disinfected and safe for its intended use. In an embodiment, the self-cleaning mechanisms may incorporate backflow washing and periodic dosing of UV, chlorine, or ozone to clean components and maintain optimal performance.

In an embodiment, the system may include an adaptive lighting and configurable camera system, which adjusts based on environmental conditions to ensure high-quality image capture across a range of monitoring scenarios. The configurable camera may ensure consistent, high-quality image capture regardless of the monitoring scenario. The lighting system may include LED arrays, UV, or infrared lights to enhance visibility, while the camera system may employ adjustable focus, zoom, and exposure settings to optimize image clarity for effective water quality analysis.

In an embodiment, the method incorporates a range of sensors, including mechanical, electromagnetic, ultrasonic flow meters, acoustic sensors, and water level sensors, for precise flow and level measurements. Acoustic sensors such as hydrophones are used for flow monitoring, with machine learning algorithms that recognize sound patterns for autonomous monitoring and prediction. The system is also capable of integrating real-time weather data and predictive forecasting to anticipate storm events and enable pre-emptive adjustments or lockdowns in stormwater and wastewater systems.

In an embodiment, the system includes a Secchi Smart Tag for consistent referencing, enabling machine learning algorithms to adjust for variability in real-world conditions. The system's opacity measurement and direct TSS ranking capabilities provide an automated assessment of effluent quality, correlating opacity with Total Suspended Solids (TSS) concentrations and estimating Biochemical Oxygen Demand (BOD) using advanced algorithms.

For a person skilled in the art, the Secchi Smart Tag is provided as an example and may include other known referencing tools, such as optical calibration discs, color reference standards, or standardized reference materials. The alternatives serve the same purpose of offering a consistent reference for accurate measurements, allowing machine learning algorithms to adjust for environmental variability and ensuring reliable, consistent water quality assessments.

Additionally, the method supports an array of modular sensors for specific liquid analysis needs, such as pH, temperature, turbidity, conductivity, dissolved oxygen, and specialized sensors for detecting contaminants like heavy metals or organic pollutants. PFAS detection can also be integrated into the system using advanced technologies such as Molecularly Imprinted Polymers (MIPs) and graphene-based nanocomposite sensors to identify trace contaminants. These sensors provide continuous, real-time monitoring, ensuring the water quality system remains adaptive, efficient, and effective across a wide range of applications.

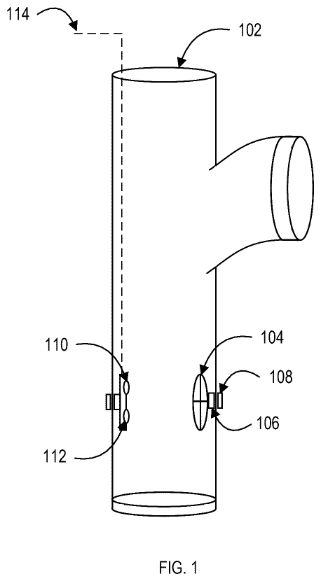

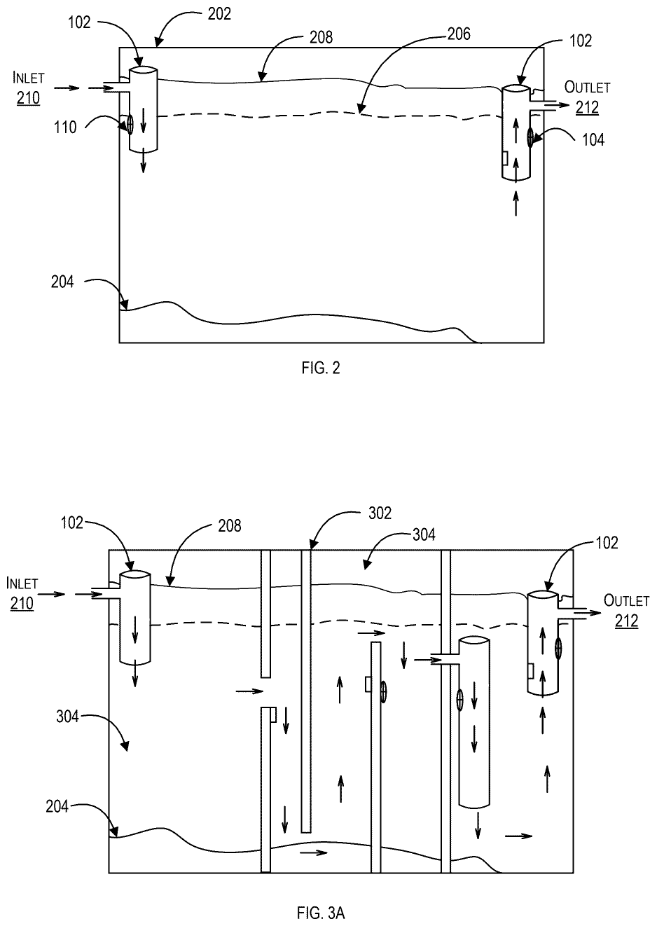

FIG. 1 illustrates an exemplary smart baffle 102 for efficient water quality monitoring, in accordance with an embodiment of the present disclosure. FIG. 2 illustrates an exemplary operation of the smart baffle 102 for monitoring wastewater in a septic tank 202, in accordance with an embodiment of the present disclosure. For the sake of brevity, FIG. 1 and FIG. 2 have been explained together.

In an embodiment, monitoring of wastewater may be performed using a smart baffle system. The smart baffle system may include the smart baffle 102. The smart baffle 102 may be a structure having a sampling chamber in fluid communication with a clarified zone of the wastewater treatment system to ensure consistent representative sampling of effluent. The sampling chamber may create optimal conditions for water quality monitoring by considering factors like lighting, fluid dynamics, fluid constituents, characteristics desired analysis, flow rate, turbulence, and particulate distribution. Septic baffles configured to integrate with pipe diameters of between 3″ and 8″ are common structures present in residential, commercial, and industrial septic systems. Some septic systems may include an inlet baffle that fluid flows down through and an inlet baffle that fluid flows up through. In some cases, septic baffles may have diameter to height ratios of about 1:2, 1:3, 1:4, 1:5, 1:6, or 2:5, where the height is the distance between an inlet and an outlet of a baffle.

In an embodiment, an inlet 210 may facilitate sewage and/or wastewater from various houses into the septic tank 202. Further, the inlet 210 may introduce wastewater at a controlled rate to minimize turbulence.

Furthermore, the inlet may include a mechanical pumping mechanism configured to pump (or draw) water samples into the internal chamber of the septic tank. In an embodiment, an outlet 212 may allow only clarified liquid to exit to drain field. Further, the outlet 212 may include integrated pumping mechanisms such as pumps or diaphragms with plumbing attachments for pumping out, holding, and discharging water samples. An anti-clogging mechanism may be utilized within the inlet 210 and outlet 212 of the septic tank 202 to reduce the risk of blockages caused by solids or debris, supporting long-term operational reliability. In an embodiment, the smart system may facilitate a gravity-driven flow mechanism to ensure an always-full sampling chamber, as shown in FIG. 2. The always-full sampling design may create stable conditions for accurate monitoring, minimizing manual intervention and supporting autonomous operation. Further, the smart baffle 102 may be a wall-mounted configuration positioned in the opening of a wall (as shown in FIG. 2) or installed along the sides of the septic tank 202. Such configuration may partially block fluid flow while permitting consistent movement between chambers, creating an effective sampling zone.

In an embodiment, the smart baffle system may facilitate monitoring stratification of zones (or layers) such as sludge 204, liquid effluent 206, and scum 208 within the septic tanks 202. Further, the smart baffle system may facilitate in precise monitoring of each layer to ensure accurate water quality assessments. In an embodiment, the smart baffle 102 may include inner magnets 106 and outer magnets 108 for secure attachment within a pipe or chamber of the septic tank 202. The inner magnets 106 and outer magnets 108 may ensure proper alignment, facilitate easy installation and removal, and allow the monitoring components to be retrofitted into existing infrastructure. In an embodiment, the smart baffle 102 may include modular ports and attachment points to enable connection with different plumbing configurations and to transform the canister into a smart baffle.

In an embodiment, the smart baffle 102 may include one or more sensors 110. The one or more sensors 110 (hereforth also termed as sensors 110) may include imaging sensors, optical sensors, pH sensors, a turbidity sensor, a dissolved oxygen sensor, and location-aware devices. Further, the sensors 110 may be strategically positioned to monitor critical water quality parameters such as opacity, turbidity, depth of solids, and chemical composition, ensuring accurate and continuous analysis of effluent quality.

In an embodiment, the sensors 110 may include radar, sonar, or chirp-based devices to measure sludge depth and scum thickness. The sensors 110 may capture real-time data on the distribution of solids and liquids within the septic tank, enabling early detection of anomalies. In an embodiment, the sensors 110 may include flow measurement sensors such as, mechanical sensors, electromagnetic sensors, ultrasonic flow meters, differential pressor sensors, acoustic sensors, doppler sensors, magnetic flow meters, and thermal mass flow meters.

In an embodiment, the imaging sensors, for visual monitoring of effluent layers, may be positioned strategically to capture the middle liquid layer, providing insights into turbidity, suspended solids correlating with Biochemical Oxygen Demand (BOD), and potential contaminants.

In an embodiment, the smart baffle 102 may include a Secchi disk 104 with a scan tag. The Secchi disk 104 may facilitate a manual cross-check for digital data and aid in training machine-learning algorithms for more precise readings. Further, the Secchi disk 104 may provide a consistent visual reference for turbidity measurements, while the scan tag may enhance imaging precision. Furthermore, the Secchi disk 104 may facilitate ranking Total Suspended Solids (TSS) present in the septic water and/or wastewater. In an embodiment, anti-microbial and anti-stick coatings may be applied to the surfaces of the sensors 110 and the Secchi disk 104. The coatings may prevent biofilm accumulation, reduce maintenance needs and ensuring long-term operational reliability. In an embodiment, the smart baffle 102 may include lighting systems 112, such as LEDs, infrared, etc., to illuminate the sampling area. The illumination may ensure clear and precise data capture under diverse environmental conditions. The lighting may improve visibility under varying conditions, including turbid or low-light environments. In an embodiment, the smart baffle may include reflective surfaces to optimize image capture under varied environmental conditions.

In an embodiment, the smart baffle system may include a power and communication line 114 connected to a control box. The power and communication line 114 may facilitate the transmission of sensor data to remote devices via Wi-Fi or other wireless protocols, supporting real-time monitoring and reporting. Communication network may include wired connections, wireless connections, and remote monitoring functionalities. Wireless connections may include Bluetooth, Wi-Fi, LoRaWAN, and/or cellular networks. A centralized database may be integrated with the wireless communication modules to transmit real-time data on sludge, liquid, and scum layers. The transmitted data may serve as input for machine learning models, such as a random forest algorithm, to analyze and predict variations in the layers based on environmental conditions. For example, the data collected from various sensors monitoring the sludge, liquid effluent, and scum layers may be used by the random forest algorithm to identify patterns and relationships between different environmental factors, such as temperature, flow rate, or chemical composition. The model may process historical and real-time data to detect subtle trends in the water quality layers, predict potential shifts in layer composition, and forecast the need for maintenance or adjustments in treatment processes.

Through training on a diverse set of data, the random forest algorithm may enhance its predictive capabilities, enabling more accurate forecasts of changes in effluent characteristics due to environmental changes or operational adjustments. For example, the random forest algorithm may facilitate identifying conditions that may lead to clogging, biofilm buildup, or the potential for system failure, allowing operators to intervene proactively. Further, the random forest algorithm may continuously improve as more data is collected, refining its predictions and providing more targeted recommendations for system optimization and maintenance. Additional systems and methods are described in U.S. Patent Application No. 2024/0279092, which shares inventorship with the present application and is hereby incorporated herein by reference in its entirety.

In an embodiment, a smart baffle system may facilitate inline installation of smart baffle 102 within septic systems and wastewater treatment systems. The smart baffle may offer effective autonomous monitoring capabilities with minimal operational disruption. Gravity-driven upward flow may be utilized to ensure consistent and reliable sampling. In an embodiment, the modular nature of the smart baffle system may facilitate customized retrofitting. The sensors and sampling chambers can be added or replaced without significant modifications to existing infrastructure setups.

In an embodiment, the smart baffle system may include specific implementations to suit various applications. For example, an outlet baffle with imaging sensors may be constructed from 2-6″ Schedule 20-80 PVC or equivalent materials. A high-frequency locator may be integrated to enable easy identification of the baffle in outdoor or underground installations. In an embodiment, the modular design of the smart baffle 102 may ensure compatibility with diverse applications, such as agricultural runoff monitoring setups and domestic, commercial, and industrial septic tanks and wastewater systems. In a particular embodiment, the smart baffle is applied to a domestic wastewater system serving a single home or a community of homes. In that embodiment, the smart baffle may comprise, for example, a 3″ or 4″ diameter baffle configured to integrate with a domestic septic system having that size outflow. The smart baffle may be placed in the septic system where an otherwise non-smart baffle would be. For instance, the smart baffle may provide for gravity-driven downward flow into a septic tank or gravity-driven upward flow out of the septic tank, where ‘upward flow’ means that an outlet of the smart baffle is above an inlet of the smart baffle, and ‘downward flow’ means that the outlet is below the inlet. The gravity-driven flow may be vertical or angled (i.e., within 10 degrees of vertical, within 30 degrees of vertical, within 45 degrees of vertical, etc.). The interior of the smart baffle may include a number of sensors configured to measure, test, or otherwise monitor wastewater flowing through the smart baffle.

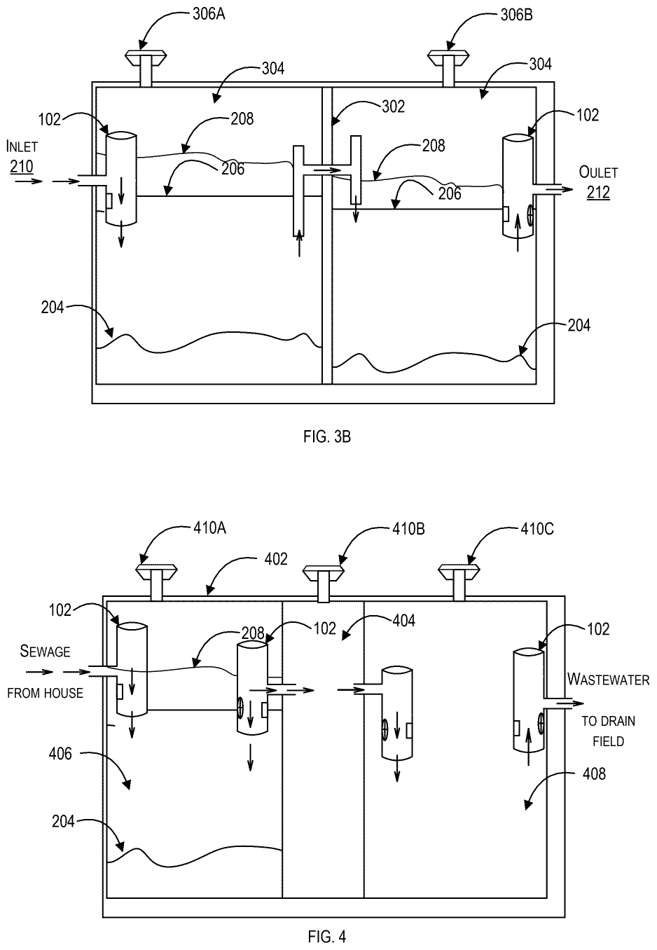

FIG. 3A and FIG. 3B illustrate exemplary operations of the smart baffle 102 for monitoring wastewater in septic tanks having chamber baffle walls 302, in accordance with an embodiment of the present disclosure. In an embodiment, the smart baffle system may include a baffle structure that partially obstructs and directs the flow of liquid, thus defining a dedicated area for sampling. Integrated sensors may enable autonomous monitoring, data capture, and analysis. The design of the baffle structure may vary, incorporating features such as grooves, holes, conduits, channels, plates, walls, screens, meshes, or balls, which may act as part of a liquid flow, distribution, or retention system.

In an embodiment, the baffle walls 302 may include non-cylindrical elements (or structures) to provide enhanced flow regulation and versatility. The non-cylindrical elements may include flat or rectangular plates that may offer uniform flow control over the liquid body. Further, the non-cylindrical elements may be placed horizontally or vertically within the system to partition the flow, directing it to specific areas while minimizing turbulence. Furthermore, the non-cylindrical elements plates may act as a solid barrier, separating two different liquid phases (such as sludge and effluent), enhancing the overall separation efficiency in wastewater treatment.

In an embodiment, the baffle walls 302 may include rectangular or square panels for precise control over the flow distribution. The rectangular or square panels may be placed strategically within the baffle structure to divide the flow into multiple smaller channels, helping to better regulate the liquid's movement through the system. In an embodiment, the panels may be perforated or solid, depending on the need for particle retention or flow regulation. The larger surface area of the rectangular or square shape may offer more opportunities to manipulate and control liquid flow compared to circular structures. The perforated or slotted walls may include a series of holes or slots that allow liquids to pass through while retaining larger particles. The holes and/or slots may facilitate separating solid particles from liquids, as the perforations provide controlled openings through which liquid can flow, while solids are left behind.

In an embodiment, the baffle walls 302 may include barrier-like walls to redirect or restrict the movement of liquid to specific areas, creating a flow path that can enhance the efficiency of treatment processes. The barrier walls may be solid and/or textured. Further, the barrier walls may be placed between different sections of the septic tank or wastewater treatment system to ensure that liquids pass through various treatment stages, each with its specific flow dynamics. In an embodiment, partitioning plates may be utilized to create smaller, more defined sections within the tank or treatment unit. The partitioning plates may be used to section off specific areas for individual processes, such as separation or filtration. Further, the partitioning plates may enhance flow management in systems with diverse phases of liquid treatment, ensuring that each phase receives optimal attention based on its specific needs.

In an embodiment, the baffle walls 302 may include mesh-like barriers made from woven materials or metal wire for creating a permeable surface that allows liquid to pass while trapping larger particles and debris. The mesh structure may be designed with varying hole sizes, allowing for fine control over particle retention. Mesh barriers may ensure that the larger solid particles do not pass through certain sections of the tank, thus improving the separation of solid and liquid phases and increasing the efficiency of wastewater treatment. Further, the mesh barriers facilitate for easier cleaning and maintenance compared to solid barriers.

In an embodiment, the baffle elements (or structures) may be used in combination to optimize the flow characteristics of wastewater in septic tanks or other treatment systems. By varying the design and layout of the elements, the system may be tailored to meet specific treatment requirements, such as solid-liquid separation, flow direction, or effluent sampling.

In an embodiment, the chamber baffle walls 302 may optimize wastewater monitoring by creating distinct zones within the septic tanks, including sludge 204, liquid effluent 206 (also termed as liquid 206), and scum 208. The zones may ensure precise sampling and analysis of each layer. Further, the chamber baffle walls 302 may direct the wastewater flow while reducing turbulence. Furthermore, the chamber baffle walls 302 may stabilize the separation of layers, improving the accuracy of sensor measurements. Moreover, the chamber baffle walls 302 may be constructed from non-corrosive materials, ensuring durability in harsh wastewater environments. Usages of such materials may reduce maintenance costs and extend the operation lifespan of the baffle system.

In an embodiment, the chamber baffle walls 302 may be coated or treated with anti-fouling or anti-microbial coatings to prevent the accumulation of biofilms, organic matter, and other contaminants that commonly build up in wastewater environments. The coatings or treatments may be applied to the surfaces of the baffle walls 302 to create a protective barrier that inhibits microbial growth, which can otherwise lead to reduced sensor accuracy and clogging. The anti-fouling coatings may resist the attachment of solids and microbial organisms, ensuring that the surfaces of the baffle walls 302 remain free from obstructions that could disrupt fluid flow or interfere with sample collection. Anti-microbial treatments, on the other hand, actively combat the growth of bacteria, algae, and other microorganisms that can form biofilms on the walls, thereby preserving the efficiency of the system over time.

In an embodiment, the coatings and/or treatments may significantly enhance the longevity and operational effectiveness of the baffle system. The coatings and/or treatments may help maintain the accuracy of sensor readings by preventing the build-up of debris. Further, the coatings and/or treatments may reduce the need for frequent maintenance and cleaning, which may lower the overall maintenance costs, ensuring that the baffle system remains operational for extended periods, even in harsh wastewater conditions, and that it continues to provide reliable and accurate water quality measurements.

In an embodiment, the inspection pipe 304 may provide access for manual observations and sample collection. In an embodiment, the baffle walls 302 may incorporate the sensors 110 to monitor critical metrics such as turbidity, flow rate, and layer thickness. The strategic placement of the sensors 110 may ensure consistent data collection across varied flow conditions.

In an embodiment, a modular configuration of the baffle system may be utilized for quality monitoring using the baffle system. The modular configuration may facilitate operators and/or users to adjust the size and placement of the smart baffle 102 based on specific application requirements. Further, the modular configuration may facilitate the installation of smart baffles in residential, industrial, and municipal settings. In an embodiment, the chamber baffle walls 302 may include flow optimization features, such as vortex reduction channels and laminar flow paths. In an embodiment, the baffle system may facilitate decentralized monitoring networks, where multiple units aggregate localized data into a central system to improve system-wide visibility and predictive maintenance.

In an embodiment, chamber(s) 304 of the septic tanks may be facilitated to autonomously analyze and monitor the flow and quality of liquid within the baffle system. The chamber(s) 304 may include the chamber baffle walls 302 and/or components directly installed within its structure, making the chamber itself the sampling area. Even distribution of liquid, measurement of flow rates, and liquid analysis may be performed within the chamber(s) 304 to optimize performance.

In an embodiment, the chamber(s) 304 may function as a dosing unit to deliver precise amounts of treatment agents. Further, the chamber(s) 304 may maintain equal loading of all drain field lines in residential septic systems. Furthermore, the chamber(s) 304 may also include control valves or mechanical levelers to adjust and redistribute flow evenly, preventing overloading of any specific lines. A maintenance alert may be triggered by the baffle system upon detecting imbalances or operational issues, facilitating early intervention and reducing system failure risks. Additionally, the chamber may include hydrophones to provide early warnings by detecting abnormal flow patterns, equipment noises, and/or infiltration/exfiltration. The hydrophones may identify unusual flow conditions, such as irregular velocities, turbulence, or changes in flow direction, which may indicate blockages, pipe malfunctions, or other issues. Further, the hydrophones may detect mechanical noises, such as those from failing pumps, valves, or other components, allowing for early identification of equipment failures. Furthermore, the hydrophones may monitor for signs of infiltration, where external water sources unintentionally enter the system, or exfiltration, where wastewater leaks out of the system, signaling potential breaches or seal failures. Moreover, the hydrophones may enhance maintenance predictability, allow for timely intervention, and improve overall system reliability by addressing potential problems before they escalate.

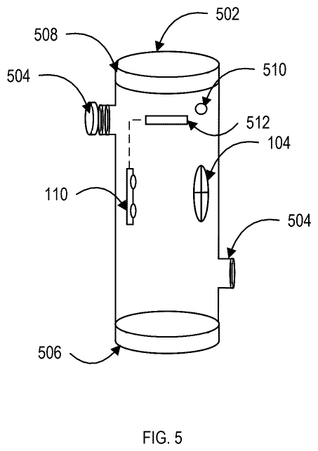

FIG. 4 illustrates an aerobic treatment system having the baffle system to optimize wastewater quality, in accordance with an embodiment of the present disclosure. In an embodiment, the aerobic treatment system may combine physical separation, biological treatment, and clarification in a modular design to optimize the wastewater quality of septic tank 402. Further, the aerobic treatment system may include an aeration chamber 404 to separate wastewater into sludge 204, liquid 206, and scum 208 layers. The one or more sensors 110 of the baffle system may monitor the thickness of these layers, ensuring efficient operation and alignment. The baffle system, retrofitted into the aerobic treatment system, may analyze the sensor data in real-time to optimize the wastewater quality in the aerobic treatment system.

In an embodiment, based on the analysis of the baffle system, the aeration chamber 404 may introduce oxygen into a layer of the liquid 206 to promote biological activity. The dissolved oxygen (DO) sensors of the sensor 110 within the smart baffle 102, may monitor aeration levels in real time, ensuring that biological activity is maintained at optimal levels for the breakdown of organic pollutants. Furthermore, the smart baffles 102 may support flow stabilization within the chamber, preventing turbulence and promoting even oxygen dispersal. In an embodiment, the clarifier chamber 406 may utilize the smart baffles 102 to facilitate the settling of solids. The strategic placement of smart baffles 102 within the clarifier chamber may enhance the sedimentation process, ensuring only clarified liquid exits the aerobic treatment system. The clarifier chamber 406 may integrate imaging sensors to assess effluent clarity. By combining flow optimization with advanced monitoring capabilities, the smart baffle system transforms the aerobic treatment system into an intelligent, data-driven solution for wastewater quality management.

In an embodiment, baffle walls within the chambers may optimize flow dynamics by minimizing turbulence and ensuring even distribution of oxygen and treatment agents. In an embodiment, the aerobic treatment system includes inspection pipes 410 for maintenance and manual observations. The inspection pipes 410 facilitate operators and/or users to verify sensor readings and make adjustments.

In an embodiment, retrofitting the baffle system with the aeration chamber may optimize the efficiency of the aerobic treatment process. By directing the flow of liquid through defined pathways, the baffle system may ensure that the liquid 206 layer is evenly exposed to the oxygen introduced by the aeration chamber 404. The controlled exposure may maximize the contact between oxygen and organic matter, thereby promoting optimal biological activity and accelerating the breakdown of pollutants. The smart baffles 102 may also stabilize flow dynamics, reducing turbulence that could disrupt the separation of wastewater layers or interfere with biological processes. Further, the baffle system's real-time monitoring capabilities may enable precise adjustments to aeration levels based on sensor data, maintaining the dissolved oxygen at ideal concentrations. The synergy between the baffle system and the aeration process may ensure a balanced and efficient treatment cycle, enhancing overall wastewater quality.

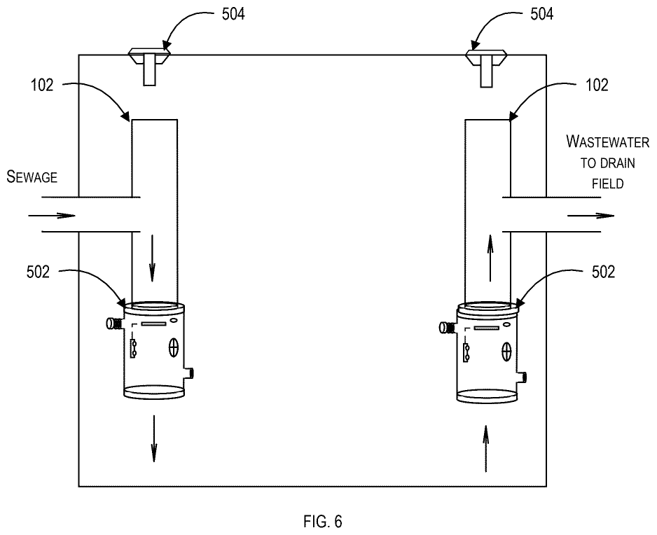

FIG. 5 illustrates exemplary Smart Inline Networked Canister (SINC) 502, in accordance with an embodiment of the present disclosure. FIG. 6 illustrates an exemplary operation of a removable top and bottom of the SINC 502, in accordance with an embodiment of the present disclosure. For the sake of brevity FIG. 5 and FIG. 6 have been explained together. The SINC 502 may be a multipurpose canister or chamber adaptable for sampling in diverse configurations, including inline, retrofitted, plumbed, submerged, or mobile monitoring scenarios. Further, the SINC 502 may enable efficient sampling and monitoring in wastewater treatment plants, agricultural runoff systems, and residential or industrial setups.

In an embodiment, the SINC 502 may include modular ports 504 that facilitate the SINC 502 t be attached to pipes, pumps, or gravity-driven flow systems. The ports 504 may enable versatile integration into various setups, such as wastewater treatment plants or agricultural runoff monitoring systems. Further, the ports 504 may facilitate versatile integration and enable the retrofitting of existing infrastructure without significant modifications. In an embodiment, knockouts or attachments may accommodate plumbing configurations, lighting, and imaging systems, enhancing their adaptability.

In an embodiment, the SINC 502 may be equipped with a removable bottom 506 and a removable top cover 508 (hereforth also together termed as removable components), simplifying maintenance by allowing easy access to internal components such as sensors or dispensers. The removable components may facilitate easy access to internal components such as sensors or dispensers during the installation and maintenance process. The removable components may allow for straightforward integration into an existing fluid flow path, ensuring seamless operation. Further, the removable components may provide an added benefit for cleaning, as they can be easily detached quickly for thorough washing and sanitation, ensuring the components remain hygienic and operational with minimal effort.

In an embodiment, the SINC 502 with removable top 508 and removable bottom 506 may be set up with various systems for efficient water treatment. For example, it may be integrated with a dishwasher setup, allowing the system to treat the wastewater generated by the dishwasher. This integration ensures effective water management and facilitates the reuse of treated water, promoting sustainability in water usage.

In an embodiment, the SINC 502 may include a chemical dispenser 510 for introducing treatment agents such as chlorine, disinfectants, or pH adjusters directly into the water flow. The chemical dispenser 510 may facilitate precise dosing, ensuring that the amount of treatment agent introduced is tailored to the real-time needs of the water being treated. The precision may be achieved through integrated flow sensors or automated control mechanisms that adjust the dispensing rate based on detected water quality parameters. Further, the chemical dispenser 510 may include a reservoir for storing multiple types of treatment agents, allowing for dynamic selection and application depending on specific requirements, such as neutralizing contaminants or maintaining desired pH levels. For example, chlorine can be dispensed to address microbial contamination, while pH adjusters can be introduced to correct imbalances in acidity or alkalinity.

In an embodiment, the chemical dispenser 510 may identify the presence of specific constituents in the water, such as nitrogen, phosphorus, and other contaminants. The identification may be performed using integrated sensors or detection systems. The sensors may operate using advanced analytical techniques, such as spectrophotometry, electrochemical sensing, or reagent-based colorimetry, to detect and quantify the levels of the constituents in real time. In an embodiment, the sensors may analyze water samples in real time and trigger the release of appropriate treatment agents to mitigate the detected issues. Further, the sensors may provide precise data on the concentration of contaminants, enabling dynamic adjustments to the type and quantity of treatment agents dispensed. If elevated nitrogen or phosphorus levels are detected, the chemical dispenser 510 may introduce specific neutralizing agents or coagulants to mitigate the effects of these contaminants. Thus, the chemical dispenser 510 may treat the water and prevent potential downstream effects such as algal blooms or ecological damage caused by nutrient overloads.

In an embodiment, dual functionality of the chemical dispenser 510 to detect contaminants and treat water may enhance the overall efficiency of water quality management by integrating monitoring and remediation into a single unit. The combination may reduce the need for separate equipment, simplifies system design, and allows for real-time, automated responses to changes in water quality. In an embodiment, the SINC 502 modular nature may allow for the addition or removal of chemical dispenser 510 and may ensure scalability and flexibility for varied applications.

In an embodiment, the SINC 502 may include the sensors 110 for monitoring critical water quality metrics such as turbidity, pH, and dissolved oxygen. The sensors 110 may be strategically placed within the SINC to capture accurate data. Adaptive lighting and imaging systems may enable high-resolution visual assessments of water samples, optimizing the identification of particulates and detecting changes in water quality in real time. For mobile use, the SINC 502 may include quick-connect fittings, allowing easy transport and deployment across multiple locations, such as dredging operations or stormwater systems.

In an embodiment, adaptive lighting and imaging systems may be integrated into the SINC 502 to enable high-resolution visual assessments of water samples. The visual assessments of water samples optimize the identification of particulates and changes in water quality.

In an embodiment, communication components 512 may be integrated into the SINC 502. The communication components 512 may support Wi-Fi or other wireless options for remote monitoring and data transmission, facilitating its integration into centralized networks. The communication components 512 may be powered by various sources, including solar panels, batteries, or hardwired electrical connections, ensuring reliability in diverse operational environments. Further, the SINC 502 may be constructed from non-corrosive and waterproof materials, enabling durability in harsh conditions such as submerged environments or high-chemical exposure areas.

In an embodiment, the modular nature of the SINC 502 may support scalable deployment, allowing the SINC 502 to be adapted for small-scale residential applications or large municipal systems.

In an embodiment, the SINC 502 may be retrofitted with the baffle system through modular ports. The retrofit may create a stable sampling zone, improving the accuracy of water quality measurements. In an embodiment, the retrofit may support customizable configurations, allowing users to add sensors 110 or imaging components as needed. The customization may ensure compatibility with diverse applications, from stormwater monitoring to industrial wastewater treatment. The retrofitted SINC 502 may function as a standalone portable unit or an inline device. For example, the SINC 502 may be mounted at the bottom of an existing outlet baffle or plumbed inline for continuous monitoring. It may also transform an agricultural drainage channel into a smart monitoring area, integrating seamlessly with sensors and communication modules to provide real-time oversight via mobile devices. The SINC 502 may be durable and include anti-corrosive and waterproof features, ensuring reliable operation in submerged environments or areas with high chemical exposure.

In an embodiment, the SINC 502 may facilitate gravity-driven flow mechanisms, ensuring consistent sample collection without mechanical intervention. In an embodiment, the SINC can be equipped with backflow washing mechanisms, enabling self-cleaning and reducing maintenance needs.

In an embodiment, SINC 502 may enhance water quality monitoring and may further provide a versatile platform for advanced treatment interventions. By integrating modular components such as chemical dispensers 510, adaptive lighting 112, and imaging systems, the SINC 502 may ensure precise and real-time assessments of water quality while simultaneously enabling proactive treatment actions. Additionally, the combination of imaging systems and strategically placed sensors 110 may ensure accurate detection and analysis of contaminants, allowing for timely adjustments in treatment processes. The blend of monitoring and intervention capabilities positions the SINC 502 as an essential tool for both preventive maintenance and active water management across a range of environments.

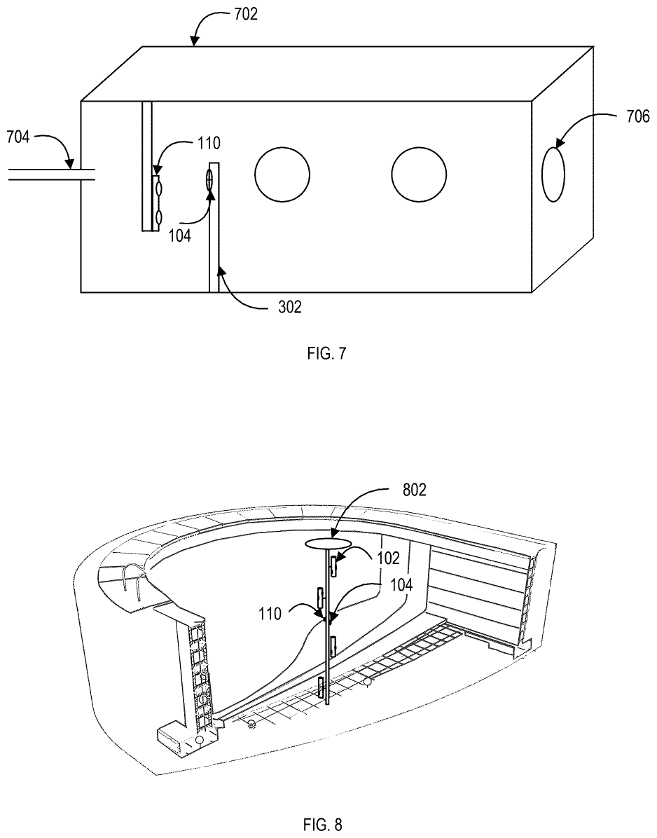

FIG. 7 illustrates an exemplary Smart Distribution Box 702 (SMWT D-Box 702) managing the flow of wastewater, in accordance with an embodiment of the present disclosure. In an embodiment, the SMWT D-Box 702 (hereforth also termed as D-box 702) may manage the flow of wastewater and provide real-time monitoring of distribution systems. The D-Box 702 may include baffle walls 302 to guide effluent flow efficiently, ensuring even distribution across downstream systems such as drain fields. Further, the baffle walls 302 may create optimized sampling areas within the D-Box 702. The sampling areas may be strategically treated with anti-fouling agents or coatings to prevent biofouling. The treated sampling areas help maintain the accuracy of sensor readings, reduce maintenance requirements, and ensure consistent water quality monitoring and flow management.

In an embodiment, the D-Box 702 may be equipped with inlets 704 and outlets 706 configured to prevent clogging and maintain consistent flow. In an embodiment, the D-Box 702 may be retrofitted into existing distribution systems, upgrading conventional setups with smart monitoring capabilities. Further, the D-Box 702 may support real-time data transmission, enabling remote monitoring and control via wireless communication modules.

In an embodiment, the modular design of the D-box 702 may facilitate scalable deployment, making the D-Box 702 suitable for residential, commercial, and municipal wastewater systems. In an embodiment, a centralized mapping platform may provide a visual overview of distribution networks for maintenance and planning purposes. In an embodiment, the D-Box 702 may include the one or more sensors 110 to monitor water quality metrics like turbidity and flow rate. The sensors 110 may provide critical data for optimizing distribution and ensuring compliance with environmental standards. Further the sensors 110 may include location-aware sensors to facilitate precise monitoring of specific zones within the D-Box 702. The location-aware sensors may track the exact location of anomalies such as flow imbalances, sediment accumulation, or localized water quality deviations. By correlating water quality data with specific locations, the D-box 702 may facilitate targeted diagnostics and interventions, such as directing cleaning or treatment efforts to affected areas without disrupting the entire system.

In an embodiment, the modular design of the D-Box 702 may facilitate seamless retrofitting into existing distribution systems, transforming traditional setups into smart, data-driven networks without significant structural changes. The inlets 704 and outlets 706 may prevent clogging and maintain a consistent flow and may ensure that the D-Box 702 operates even under high-demand conditions. Remote monitoring capabilities enabled by wireless communication modules may further enhance the system's utility, allowing operators to access real-time data and adjust remotely. The combination of advanced features may make the D-Box 702 an indispensable tool for modern wastewater management across residential, commercial, and municipal applications.

FIG. 8 illustrates an exemplary solar-powered buoy system 802 for remote water quality monitoring and management, in accordance with an embodiment of the present disclosure. In an embodiment, the buoy system 802 may be deployed in diverse environments, from swimming pools and hot tubs to natural water bodies, demonstrating its versatility and adaptability. Further, the buoy system 802 may include solar panels to power the incorporated sensors 110 and communication modules, ensuring continuous operation, even in remote locations. Further, the sensors 110 may monitor parameters such as turbidity, pH, chlorine/bromine levels, total dissolved solids (TDS), oxidation-reduction potential (ORP), and alkalinity. In an embodiment, the buoy system 802 may provide real-time monitoring capability and may ensure optimal water quality, enhance user safety, reduce maintenance efforts, and prolong equipment lifespan.

In an embodiment, the buoy system 802 includes adaptive lighting and imaging systems to capture high-resolution visual data, enhancing the detection of contaminants and supporting automated water quality assessments. Further, the buoy system 802 may enable the detection of algae growth, biofilm formation, or debris accumulation, facilitating automated water quality assessments. Data collected by the sensors 110 and imaging systems may be processed in real-time using machine learning algorithms, allowing the buoy system 802 to perform advanced functions such as precise chemical dosing and equipment control. This reduces the need for manual intervention, ensuring consistent water quality maintenance.

In an embodiment, the buoy system 802 includes the baffle system having the baffle 102 and/or may incorporate SINC 502 for monitoring water quality. The baffle system may create stable sampling zones for accurate analysis of water parameters, while the SINC 502 may support additional sensor integration and chemical treatment capabilities, making the buoy adaptable to diverse water management scenarios.

In an embodiment, the buoy system 802 may be constructed with non-corrosive materials to withstand harsh aquatic conditions, ensuring durability and long-term reliability. Further, the buoy system may include self-cleaning mechanisms and automated filters, which help maintain sensor accuracy and filtration efficiency. Furthermore, the buoy system 802 may support remote monitoring and control, transmitting data wirelessly to a central platform via Wi-Fi or cellular networks.

In an embodiment, the solar-powered buoy system 802 deployed in swimming pools offers an advanced solution for maintaining optimal water quality with minimal manual effort. The buoy system 802 continuously monitors critical parameters, using the sensors 110, including pH, chlorine/bromine levels, total dissolved solids (TDS), oxidation-reduction potential (ORP), and alkalinity, ensuring compliance with safety and health standards. Adaptive imaging systems enhance monitoring by detecting contaminants, algae growth, or biofilm formation, allowing for timely interventions. The system's ability to process collected data in real-time through machine learning algorithms enables automated functions such as precise chemical dosing and filtration adjustments, ensuring consistent water quality and extending the lifespan of pool equipment.

In an embodiment, integration/retrofitting of baffle systems 102 and/or SINC 502 modules with the buoy system may enhance performance in swimming pool applications. The baffle system may create stable sampling zones for accurate analysis of water parameters, while the SINC 502 may provide additional chemical treatment capabilities, adapting seamlessly to varied maintenance needs. Constructed from non-corrosive materials, the buoy system may withstand the chemical exposure typical of pool environments and may incorporate self-cleaning mechanisms to reduce maintenance requirements. Wireless connectivity may allow for remote monitoring and control, providing users with real-time updates and automated management through centralized platforms, making the buoy system a versatile and efficient solution for residential and commercial swimming pools.

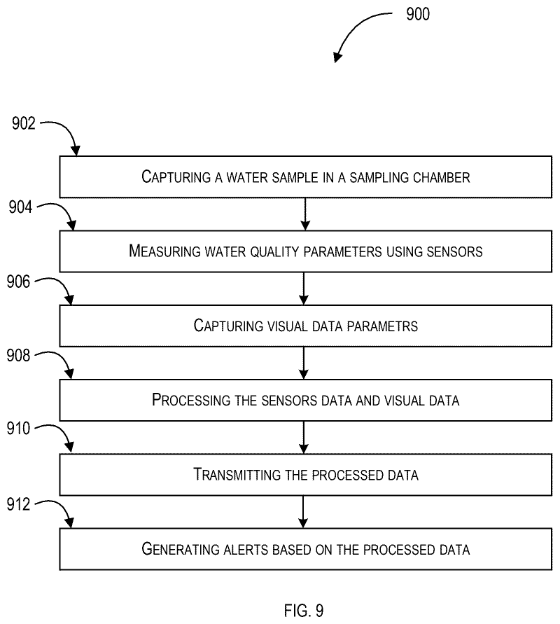

FIG. 9 illustrates a flowchart 900 of a method for monitoring water quality, in accordance with an embodiment of the present disclosure. At first, a water sample is captured in a sampling chamber, at step 902. The sampling chamber may be configured to receive a continuous or on-demand flow of water. Further, the sampling chamber may be an inline canister, a portable device, or a retrofit attachment integrated into existing water infrastructure. Furthermore, the sampling chamber may include inlet and outlet ports to control fluid dynamics, ensuring laminar flow and minimizing turbulence. The controlled fluid dynamics may enhance the accuracy of measurements by providing a stable environment for sampling. In an embodiment, the sampling chamber may incorporate flow control mechanisms, such as baffles or valves, to regulate the flow rate, ensuring that the water sample represents the overall water body. In gravity-driven systems, the sampling chamber may remain consistently filled without mechanical intervention. Alternatively, in environments with low flow, a mechanical pump may draw water into the chamber. In an embodiment, the canister may be configured with detachable components, including interchangeable end caps, to facilitate different configurations and use cases. Further, the canister may be adaptable for use in various water quality environments, including septic systems, stormwater management systems, agricultural runoff areas, pools, hot tubs, and industrial wastewater facilities.

At step 904, the method may include measuring various water quality parameters using sensors disposed of within the sampling chamber. The sensors may detect parameters such as pH, turbidity, dissolved oxygen, temperature, and conductivity. In an embodiment, multi-parameter sensors may be employed to detect chemical, physical, and biological properties simultaneously. For instance, pH sensors may use electrochemical electrodes to measure hydrogen ion concentration, while turbidity sensors may use optical scattering techniques to assess water clarity. Dissolved oxygen sensors may operate using either electrochemical or optical methods to monitor oxygen levels critical for aquatic ecosystems. Further, sensors for detecting specific contaminants, such as nitrates, phosphates, or heavy metals, may be incorporated to enable targeted analysis in industrial or agricultural applications. In an embodiment, sensors may be equipped with anti-fouling coatings to prevent biofilm buildup and ensure consistent accuracy over extended periods.

At step 906, the method may include capturing visual data of the water sample using imaging mechanisms positioned within or adjacent to the sampling chamber. The imaging mechanisms may include a high-resolution camera with adaptive optics to capture detailed images of the water sample. The imaging mechanism may provide additional insights into water quality by detecting particulate matter, color changes, or microbial growth, which may not be captured by conventional sensors alone. Lighting systems, including LEDs, ultraviolet (UV), or infrared (IR) lights, may be integrated to enhance image clarity under varying environmental conditions, such as low light or high turbidity.

At step 908, the method may include processing the data collected by the sensors and imaging mechanisms to analyze water quality and detect deviations from predefined thresholds. The method may include utilizing machine learning algorithms to compare real-time data against historical baselines, enabling the detection of trends, anomalies, or sudden changes in water quality. Further, the method may apply advanced data analytics to identify patterns indicative of contamination, equipment failure, or environmental events. Furthermore, the method may include integrating self-calibration routines to ensure ongoing accuracy by periodically validating sensor outputs against reference standards.

At step 910, the method may include transmitting the processed data to a remote device or centralized database via a communication module. The transmitted data may include both raw sensor readings and processed analytics, providing operators with comprehensive insights into water quality conditions.

At step 912, the method may include generating alerts and/or reports based on processed data to inform operators of deviations from predefined water quality thresholds. Alerts may be automatically triggered when critical parameters exceed safe limits, notifying operators via email, SMS, or mobile applications. In an embodiment, the alerts may include actionable insights, such as recommended maintenance actions or potential contamination sources. Reports may be generated periodically, summarizing water quality trends, maintenance activities, and regulatory compliance status.

In an embodiment, the method may include providing visual dashboards that display real-time data, historical trends, and key performance indicators, enabling users to make informed decisions quickly.

The embodiments of the present invention are merely exemplary and are not intended to limit the scope of the invention in any way. The monitoring system may include a variety of configurations and applications, each designed to improve water quality monitoring and management in different environments. One such embodiment is a retrofit kit for industrial discharge monitoring, which converts existing industrial discharge systems into smart, monitored units. This system may feature modular sensors designed to monitor critical water quality parameters such as Chemical Oxygen Demand (COD), pH, conductivity, heavy metals, and temperature. The communication capabilities of the system may include options such as LoRaWAN, Wi-Fi, or wired connections, ensuring flexible data transmission. Additionally, the adaptable mounting options of the system allow for easy integration into existing infrastructures, while anti-corrosion treatments ensure the durability and reliability of the components in harsh industrial environments.

Another embodiment may include a portable canister for monitoring agricultural runoff and controlling nutrient levels. This canister may be equipped with specialized nutrient sensors for monitoring key substances such as nitrate, phosphate, and ammonium. The canister can also include a dispensing unit for neutralizing agents to manage nutrient levels effectively. Designed for deployment at runoff points, the canister is equipped with quick-connect fittings to allow for easy installation and removal. The system may transmit collected data via Bluetooth or Wi-Fi, providing real-time information on nutrient levels and helping to mitigate the environmental impacts of agricultural runoff.

Additionally, the system may include an industrial effluent quality monitoring and control unit designed to monitor the quality of effluent outflows in industrial settings. This embodiment may feature sensors for pH, COD, temperature, and turbidity, along with a flow control valve to regulate discharge rates. The system may be modular, allowing for easy retrofitting of existing equipment, and the data may be transmitted to a cloud-based dashboard for remote monitoring and analysis.

For stormwater management, the invention may include a stormwater smart basin monitoring system, which can retrofit stormwater basins to monitor real-time water quality and control overflow. The system may include adaptive overflow valves and sensors for turbidity, heavy metals, and pH, with the data transmitted via LoRaWAN. Additionally, the system may be solar-powered, offering autonomous operation in remote areas.

In another embodiment, the invention may be adapted for stormwater flood prevention and quality monitoring. This system integrates into urban stormwater systems to both prevent flooding and improve water quality. It may feature sensors for water level and turbidity, along with an adaptive valve to adjust the flow of water. The system transmits data to a cloud-based platform for centralized monitoring and is solar-powered for autonomous operation.

The invention may also include a stormwater sediment capture and monitoring module, which captures sediments from urban runoff while monitoring water quality in real time. This system may include a sediment trap with turbidity sensors and a self-cleaning backflush mechanism, and the collected data is transmitted to municipal systems for analysis and reporting.

For septic system management, the system may include a smart distribution box retrofit system. This retrofit kit is designed to convert traditional distribution boxes into monitored units, allowing for more efficient management of effluent flow to drain fields. The system may include flow sensors and smart valves to balance load across drain field laterals, and it can be retrofitted with a communication module for remote monitoring and alerts.

The system may also be designed for specific applications such as dredging operations. A portable canister for dredging operations may monitor turbidity and pollutant levels in underwater conditions. This embodiment may feature pressure-resistant sensors for use in submerged environments and GPS for location-based data collection, which can be attached to dredging equipment for continuous monitoring.

Additionally, the system may include a smart grease trap monitoring system for existing grease traps. This retrofit system provides real-time monitoring of grease buildup using ultrasonic or capacitive sensors to measure grease thickness. The system may also include a communication module for remote alerts, as well as integrated lighting and imaging components for assessing the contents of the trap. The system is powered by both battery and solar energy, ensuring autonomous operation and reducing the need for manual maintenance.

The system may also be adapted for cooling tower water quality management. This embodiment continuously monitors water quality parameters such as pH, conductivity, and temperature to prevent scaling and corrosion. The system can trigger automatic dosing of anti-corrosive agents or descaling chemicals as needed, helping to prolong equipment life, reduce maintenance downtime, and maintain energy efficiency in cooling towers.

Another example of the invention's versatility is in portable water quality monitoring. The system may be designed for temporary and flexible deployment in various water bodies, such as rivers, lakes, ponds, or reservoirs. Equipped with multi-parameter sensors for pH, turbidity, temperature, and specific pollutants, this portable unit is ideal for environmental assessments, emergency spill monitoring, and research purposes.

Finally, the system may be used for pathogen detection in wastewater. This embodiment integrates advanced pathogen detection capabilities, using biosensors like qPCR or LAMP to detect genetic material from viruses such as SARS-CoV-2, bacteria, and other pathogens. The data collected is transmitted to a centralized monitoring platform for early warning and proactive response, ensuring public health and safety.

Embodiments of the present disclosure method and apparatus for monitoring water quality (hereforth written as mechanism) offer significant advantages over traditional water quality monitoring systems. The mechanism provides automated, real-time monitoring and management of water quality. Unlike conventional systems that rely on manual sampling and laboratory analysis, the disclosed mechanism enables continuous monitoring, real-time data capture, and automated reporting. This automation ensures regulatory compliance, reduces human error, and minimizes administrative burdens, effectively addressing the limitations of traditional systems. Additionally, it mitigates the risk of fines and non-compliance by delivering accurate, consistent, and actionable insights into water quality.

Further, the disclosed mechanism facilitates preventive maintenance and early issue detection through advanced sensors and machine learning algorithms. Real-time anomaly detection enables proactive intervention, significantly reducing the risk of costly repairs, system downtime, or environmental contamination. By identifying issues early, the disclosed mechanism extends the lifespan of water treatment infrastructure and minimizes operational disruptions, providing a far superior approach compared to reactive maintenance methods. Another key benefit is the support for environmental sustainability by identifying contaminants early and preventing their release into natural waterways. This proactive approach aligns with sustainable water management practices, ensuring better protection for ecosystems and contributing to overall environmental health. Moreover, the system's efficient design and modular components help reduce resource waste, further promoting responsible water use.

In an embodiment, the lower labor costs and improved resource utilization facilitate property owners, service providers, and municipalities to benefit from reduced downtime and enhanced mechanism reliability, leading to substantial cost savings. The flexibility of the mechanism makes it suitable for small-scale residential applications as well as large municipal installations, offering efficiency across various scales. Further, the modular design of the disclosed mechanism makes it applicable to a wide range of settings, including residential septic systems, industrial wastewater, stormwater management, agricultural runoff, and recreational water facilities. Its ability to work in both permanent installations and portable monitoring setups ensures it is adaptable to diverse scenarios. In terms of insurance coverage, the mechanism may help mitigate risks by providing consistent monitoring and early detection of potential issues. This reduces the likelihood of costly repairs and system failures, making infrastructure like septic systems more insurable, which offers financial benefits to property owners.

In an embodiment, the system may enhance public health by ensuring that water treatment systems operate effectively, preventing contamination of drinking water sources and recreational areas. Real-time monitoring capabilities allow for rapid detection of pathogens, reducing potential health hazards and improving community health outcomes. Data-driven insights generated by continuous data collection help drive informed decision-making, optimize system design, and advance water quality technologies. Integration with centralized monitoring platforms further supports policy-making and operational strategies, allowing for continuous improvement. The mechanism's decentralized and scalable monitoring capabilities enable multiple units to function cohesively within a network, improving system-wide data visibility and enabling predictive maintenance. The decentralized approach enhances operational reliability and ensures the system is scalable for both small and large installations.

Additionally, the mechanism integrates and retrofits existing infrastructure and offers a cost-effective way to upgrade traditional water management systems into intelligent, automated systems. This flexible integration ensures improved operational efficiency and regulatory compliance without the need for major modifications to existing infrastructure. Overall, the disclosed mechanism provides enhanced operational efficiency, broader application potential, and improved water quality management.

While embodiments of the present disclosure have been illustrated and described, it will be clear that the disclosure is not limited to these embodiments only. Numerous modifications, changes, variations, substitutions, and equivalents will be apparent to those skilled in the art, without departing from the spirit and scope of the disclosure, as described in the claims.

Thus, it will be appreciated by those of ordinary skill in the art that the diagrams, schematics, illustrations, and the like represent conceptual views or processes illustrating systems and methods embodying this disclosure. The functions of the various elements shown in the figures may be provided through the use of dedicated hardware as well as hardware capable of executing associated software. Similarly, any switches shown in the figures are conceptual only. Their function may be carried out through the operation of program logic, through dedicated logic, through the interaction of program control and dedicated logic, or even manually, the particular technique being selectable by the entity implementing this disclosure. Those of ordinary skill in the art further understand that the exemplary hardware, software, processes, methods, and/or operating systems described herein are for illustrative purposes and, thus, are not intended to be limited to any particular named.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously. Within the context of this document terms “coupled to” and “coupled with” are also used euphemistically to mean “communicatively coupled with” over a network, where two or more devices can exchange data with each other over the network, possibly via one or more intermediary device.

It should be apparent to those skilled in the art that many more modifications besides those already described are possible without departing from the inventive concepts herein. The inventive subject matter, therefore, is not to be restricted except in the spirit of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, utilized, or combined with other elements, components, or steps that are not expressly referenced. Where the specification claims refer to at least one of something selected from the group consisting of A, B, C .... and N, the text should be interpreted as requiring only one element from the group, not A plus N, or B plus N, etc.

While the foregoing describes various embodiments of the disclosure, other and further embodiments of the disclosure may be devised without departing from the basic scope thereof. The scope of the disclosure is determined by the claims that follow. The disclosure is not limited to the described embodiments, versions, or examples, which are included to enable a person having ordinary skill in the art to make and use the disclosure when combined with information and knowledge available to the person having ordinary skill in the art.

### Example Features

The following examples pertain to further embodiments, from which numerous permutations and configurations will be apparent.

Example 1 is a device for monitoring water quality in a septic system, the device comprising an inlet in fluid communication with the septic system, an outlet, a sampling chamber in fluid communication with the inlet and the outlet and at least partially enclosed by a body of the device, one or more sensors incorporated into the device and configured to measure one or more water quality metrics of a fluid inside the sampling chamber, and a microcontroller configured to process or transmit water quality metrics measured by the one or more sensors.

Example 2 is the device of example 1, wherein at least one of the one or more sensors is configured to measure at least one of pH, temperature, conductivity, dissolved oxygen, turbidity, and pollutant concentration of the fluid inside the sampling chamber.

Example 3 is the device of example 2, further comprising a dosing unit configured to release one or more water treatment agents based on at least one of the one or more water quality metrics.

Example 4 is the device of example 1, wherein the device is at least partially coated with a nonstick coating or an antimicrobial coating.

Example 5 is the device of example 1, wherein at least one of the one or more sensors is a hydrophone configured to monitor a flow rate of the fluid.

Example 6 is the device of example 1, further comprising one or more image capture devices configured to gather visual data depicting the fluid inside the sampling chamber, and wherein the microcontroller is further configured to produce water quality data based on the visual data.

Example 7 is the device of example 1, wherein the septic system is a residential septic system, the inlet and the outlet have diameters of between 3 inches and 6 inches, and device is configured for gravity-driven upward flow from the inlet to the outlet.

Example 8 is a water quality monitoring system, comprising a septic tank having a source and an outlet in fluid communication via an interior of the septic tank, a plurality of structural elements disposed within the septic tank configured to direct a flow of fluid between the source and the outlet, one or more sensors incorporated into the one or more structural elements and configured to monitor one or more water quality metrics of the fluid as it flows through the septic tank, a microcontroller in electrical communication with the one or more sensors and configured to process and communicate water quality data based on the one or more water quality metrics.

Example 9 is the system of example 8, wherein at least one of the one or more sensors is configured to measure at least one of pH, temperature, conductivity, dissolved oxygen, turbidity, and pollutant concentration of the fluid inside the septic tank.

Example 10 is the system of example 8, further comprising a dosing unit controlled by the microcontroller and configured to release one or more water treatment agents into the septic tank based on the water quality data.

Example 11 is the system of example 8, wherein the microcontroller is configured to issue at least one of alerts and reports based on the water quality data.

Example 12 is the system of example 8, wherein a subset of the structural elements are partially permeable structural elements that fluid can pass through.

Example 13 is the system of example 12, wherein the partially permeable structural elements contain perforations configured to extract particles from the fluid as it passes through the permeable structural elements.

Example 14 is the system of example 8 further comprising an aeration chamber configured to introduce dissolved oxygen into the fluid.

Example 15 is the system of example 8 further comprising one or more hydrophones integrated within the septic tank and configured to monitor for flow rate and mechanical noises within the septic tank.

Example 16 is a method of monitoring water quality comprising flowing a wastewater sample from a domestic wastewater system into a sampling chamber, measuring one or more water quality parameters of the wastewater sample via one or more sensors integrated into the sampling chamber, capturing visual data depicting the wastewater sample via one or more image capture devices incorporated within the sampling chamber, processing the one or more water quality parameters and the visual data via a microcontroller to produce water quality data, and flowing the wastewater sample out of the sampling chamber.

Example 17 is the method of example 16, wherein measuring one or more water quality parameters of the wastewater sample via one or more sensors comprises measuring at least one of pH, temperature, conductivity, dissolved oxygen, turbidity, and pollutant concentration of the water sample.

Example 18 is the method of example 16, further comprising capturing audio data depicting the flowing of the wastewater sample into and out of the sampling chamber and processing the audio data to produce flow rate data and mechanical event data.

Example 19 is the method of example 16, further comprising releasing one or more water treatment agents into the domestic, commercial, or industrial plumbing line based on the water quality data.

Example 20 is the method of example 16, further comprising generating and communicating one of an alert and a report based on the water quality data.

1. A device for monitoring water quality in a septic system, the device comprising:
an inlet in fluid communication with the septic system;
an outlet;
a sampling chamber in fluid communication with the inlet and the outlet and at least partially enclosed by a body of the device;
one or more sensors incorporated into the device and configured to measure one or more water quality metrics of a fluid inside the sampling chamber; and
a microcontroller configured to process or transmit water quality metrics measured by the one or more sensors.

2. The device of claim 1, wherein at least one of the one or more sensors is configured to measure at least one of pH, temperature, conductivity, dissolved oxygen, turbidity, and pollutant concentration of the fluid inside the sampling chamber.

3. The device of claim 2, further comprising a dosing unit configured to release one or more water treatment agents based on at least one of the one or more water quality metrics.

4. The device of claim 1, wherein the device is at least partially coated with a nonstick coating or an antimicrobial coating.

5. The device of claim 1, wherein at least one of the one or more sensors is a hydrophone configured to monitor a flow rate of the fluid.

6. The device of claim 1, further comprising one or more image capture devices configured to gather visual data depicting the fluid inside the sampling chamber, and wherein the microcontroller is further configured to produce water quality data based on the visual data.

7. The device of claim 1, wherein the septic system is a residential septic system, the inlet and the outlet have diameters of between 3 inches and 6 inches, and device is configured for gravity-driven upward flow from the inlet to the outlet.

8. A water quality monitoring system, comprising:
a septic tank having a source and an outlet in fluid communication via an interior of the septic tank;
a plurality of structural elements disposed within the septic tank configured to direct a flow of fluid between the source and the outlet;
one or more sensors incorporated into the one or more structural elements and configured to monitor one or more water quality metrics of the fluid as it flows through the septic tank; and
a microcontroller in electrical communication with the one or more sensors and configured to process and communicate water quality data based on the one or more water quality metrics.

9. The system of claim 8, wherein at least one of the one or more sensors is configured to measure at least one of pH, temperature, conductivity, dissolved oxygen, turbidity, and pollutant concentration of the fluid inside the septic tank.

10. The system of claim 8, further comprising a dosing unit controlled by the microcontroller and configured to release one or more water treatment agents into the septic tank based on the water quality data.

11. The system of claim 8, wherein the microcontroller is configured to issue at least one of alerts and reports based on the water quality data.

12. The system of claim 8, wherein a subset of the structural elements are partially permeable structural elements that fluid can pass through.

13. The system of claim 12, wherein the partially permeable structural elements contain perforations configured to extract particles from the fluid as it passes through the permeable structural elements.

14. The system of claim 8 further comprising an aeration chamber configured to introduce dissolved oxygen into the fluid.

15. The system of claim 8 further comprising one or more hydrophones integrated within the septic tank and configured to monitor for flow rate and mechanical noises within the septic tank.

16. A method of monitoring water quality comprising:
flowing a wastewater sample from a domestic wastewater system into a sampling chamber;
measuring one or more water quality parameters of the wastewater sample via one or more sensors integrated into the sampling chamber;
capturing visual data depicting the wastewater sample via one or more image capture devices incorporated within the sampling chamber;
processing the one or more water quality parameters and the visual data via a microcontroller to produce water quality data; and
flowing the wastewater sample out of the sampling chamber.

17. The method of claim 16, wherein measuring one or more water quality parameters of the wastewater sample via one or more sensors comprises measuring at least one of pH, temperature, conductivity, dissolved oxygen, turbidity, and pollutant concentration of the water sample.

18. The method of claim 16, further comprising capturing audio data depicting the flowing of the wastewater sample into and out of the sampling chamber and processing the audio data to produce flow rate data and mechanical event data.

19. The method of claim 16, further comprising releasing one or more water treatment agents into the domestic, commercial, or industrial plumbing line based on the water quality data.

20. The method of claim 16, further comprising generating and communicating one of an alert and a report based on the water quality data.
